(19) United States
(12) Patent Application Publication
(10) Pub. No.: US 2003/0198602 A1
(43) Pub. Date: Oct. 23, 2003

(54) Methods for pulmonary delivery of interleukin-2

(75) Inventors: Bao-Lu Chen, San Ramon, CA (US); Maninder Hora, Danville, CA (US)

Correspondence Address: Chiron Corporation, Intellectual Property - R440, P.O. Box 8097, Emeryville, CA, 94662-8097, US

(73) Assignee: Chiron Corporation, Emeryville, CA

(21) Appl. No.: 10/408,648

(22) Filed: Apr. 7, 2003

Related U.S. Application Data
(63) Continuation of application No. 09/724,810, filed on Nov. 28, 2000, now abandoned.
(60) Provisional application No. 60/173,922, filed on Dec. 30, 1999.

(51) Int. Cl. A61L 9/04; A61K 9/14; A61K 38/20

(52) U.S. Cl. 424/46; 424/85.2

(57) ABSTRACT

Methods for administering interleukin-2 (IL-2) or variants thereof via pulmonary inhalation are provided. The methods comprise preparing a pharmaceutical composition comprising IL-2 or variants thereof for subsequent delivery as an aqueous or nonaqueous solution or suspension or a dry powder form. These compositions may further comprise a surfactant in an amount sufficient to enhance absorption of the composition following pulmonary inhalation of the composition. When used in the delivery methods of the invention, these highly absorbable compositions result in enhanced bioavailability of IL-2.

### CROSS-REFERENCE TO RELATED APPLICATIONS

This application is a continuation of U.S. application Ser. No. 09/724,810, filed Nov. 28, 2000, which claims the benefit of U.S. Provisional Application No. 60/173,922, filed Dec. 30, 1999, the contents of which are herein incorporated by reference in their entirety.

### FIELD OF THE INVENTION

The present invention is related to methods of protein delivery to mammals, more specifically to delivery of interleukin-2 proteins via pulmonary inhalation and compositions useful in the methods of the invention.

### BACKGROUND OF THE INVENTION

Pulmonary inhalation provides a promising route for absorption of peptides and proteins having poor oral bioavailability due to inefficient transport across the gastrointestinal epithelium or high levels of first-pass hepatic clearance. Potential advantages of this delivery route for peptide- or protein-containing drugs include a greater extent of absorption due to an absorptive surface area of approximately 140 m² and high volume of blood (5000 ml/min in the human lung) flowing through the lungs (Hollinger (1985) in Respiratory Pharmacology and Toxicology (Saunders, Pa.), pp. 1-20). Lack of some forms of peptidase/protease activity when compared with the gastrointestinal tract and lack of first-pass hepatic metabolism of absorbed compounds adds to the potential benefits of protein drug administration via pulmonary inhalation. Interest in this delivery route has increased in recent years since a number of potential peptide- or protein-containing drugs are absorbed more efficiently from the lung than from the gastrointestinal tract (Patton and Platz (1992) Adv. Drug Del. Rev. 8:179-196; Niven (1993) Pharm. Technol. 17:72-82).

Delivery of peptide- and/or protein-containing pharmaceutical formulations via pulmonary inhalation is known, although only a few examples have been quantitatively substantiated. See, for example, Hubbard et al. (1989) Ann. Internal Med.. 3(3): 206-212 (plasma α-1-antitrypsin); Smith et al. (1989) J. Clin. Invest. 84: 1145-1154 (α-1-proteinase inhibitor). Experiments with test animals have shown that recombinant human growth hormone, when delivered by aerosol, is rapidly absorbed from the lung and produces faster growth comparable to that seen with subcutaneous injection (Oswein et al. (1990), “Aerosolization of Proteins” in Proceedings of Symposium on Respiratory Drug Delivery II (Keystone, Colo., March, 1990). Recombinant versions of the cytokines gamma interferon (IFN-γ) and tumor necrosis factor alpha (TNF-α) have also been observed in the bloodstream after aerosol administration to the lung (Debs et al. (1988) J. Immunol. 140:3482-3488). The feasibility of pulmonary delivery of granulocyte-colony stimulating factor (G-CSF) and erythropoietin (EPO) to mammals has also been demonstrated. See U.S. Pat. Nos.5,284,656 and 5,354,934, respectively. See also U.S. Pat. No. 5,997,848, where systemic delivery of insulin to a mammalian host is accomplished via inhalation of a dry powder aerosol containing insulin.

Despite these examples of protein delivery via the pulmonary route, it remains unpredictable whether a particular polypeptide can be delivered for systemic effect in such a manner. Additionally, the bioavailability of polypeptides delivered via the pulmonary route is usually quite low. For example, pulmonary administration of endothelin-1 (ET-1), a 21 amino-acid vasoconstrictor peptide produced by endothelial cells, is not as effective as intravenous administration (Braquet et al. (1989) J. Cardio. Pharm. 13, suppl. 5:143-146). Vasoactive intestinal peptide, a small polypeptide with a molecular weight of 3,450 daltons (D) which causes bronchodilation when given intravenously in animals, including humans, lacks efficacy when administered by inhalation (Barrowcliffe et al. (1986) Thorax. 41/42: 88-93).

Successful delivery of peptides or proteins into the deep lung tissues is dependent upon a number of factors. The extent of absorption within the lung tissues varies with size and structure of the polypeptide and the delivery device used, ranging from 0-95% of the dose administered. Such delivery devices include nebulizers, metered-dose inhalers, and powder inhalers. Preparation of protein or peptide-containing pharmaceutical compositions as an aqueous liquid aerosol, a nonaqueous suspension aerosol, or dry powder aerosol for pulmonary administration using these respective delivery devices can influence polypeptide stability, and hence bioavailability as well as biological activity following delivery. See Wall (1995) Drug Delivery 2:1-20; Krishnamurthy (March 1999) BioPharm., pp. 34-38). Thus, successful pulmonary delivery of a particular therapeutic protein to bring about a systemic effect cannot be anticipated in advance of animal model testing. One such model is the intratracheal (IT) technique (Niven et al. (1994) Pharm. Res. 12:1142-1149; Niven et al. (1995) Pharm. Res. 12:1889-1895). This model is predictive of pulmonary absorption of therapeutic proteins (see, for example, Patton et al. (1994) J. Controlled Release 28:79-85).

Interleukin-2 is normally administered via intravenous or subcutaneous injection. Pulmonary administration of this protein would provide an attractive noninvasive alternative to these routes of administration. Pulmonary inhalation of interleukin-2 has been demonstrated using a nebulizer to deliver an aqueous liquid formulation containing IL-2 (U.S. Pat. Nos. 5,399,341 and 5,780,012). However, pulmonary administration of polypeptides as an aerosol using the nebulizer system has been shown to denature some polypeptides (see Ip et al. (1995) J. Pharm. Sci. 84:1210-12-14 (interferon); Niven et al (1994) Int. J. Pharm. 109:17-26 (recombinant granulocyte-colony-stimulating factor); and Niven et al. (1995) Pharm. Res. 12:53-59). During the nebulization process, the polypeptide is exposed to shearing stresses that may aggravate loss of biological activity.

In view of the above, delivery of proteins at a high bioavailability remains a challenging task. Additional noninvasive methods for administering IL-2 for systemic response are needed.

### SUMMARY OF THE INVENTION

Methods for administering interleukin-2 (IL-2) or variants thereof via pulmonary inhalation are provided. The methods comprise preparing a pharmaceutical composition comprising IL-2 or variants thereof for subsequent delivery as an aqueous or nonaqueous solution or suspension or a dry powder form. Compositions for use in this method include compositions comprising stabilized monomeric IL-2 or variants thereof, compositions comprising multimeric IL-2 or variants thereof, and compositions comprising stabilized lyophilized or spray-dried IL-2 or variants thereof Each of these compositions may further comprise a surfactant in an amount sufficient to enhance absorption of the composition following pulmonary inhalation of the composition. Such compositions are referred to as highly absorbable compositions.

The invention further provides a method for enhancing bioavailability of IL-2 administered to a subject via pulmonary inhalation. The method comprises preparing an aerosol or other suitable preparation of the highly absorbable compositions disclosed herein and administering the aerosol or other suitable preparation to the subject via pulmonary inhalation.

### BRIEF DESCRIPTION OF THE DRAWINGS

FIG. 1 shows mean plasma concentration profiles of Proleukin following intratracheal (IT) (750 μg rhIL-2/animal) and subcutaneous (SC) (150 μg rhIL-2/animal) administration in rats.

FIG. 2 shows mean plasma concentration profiles of monomeric rhIL-2 with polysorbate 80 (Tween 80) following intratracheal (IT) (375 μg/animal) and subcutaneous (SC) (75 μg/animal) administration in rats.

FIG. 3 shows plasma IL-2 concentration following intratracheal (IT) (375 μg/animal), subcutaneous (SC) (150 μg/animal), or intravenous (IV) (150 μg /animal) administration of monomeric rhIL-2 with and without polysorbate 80 (Tween 80) in rats.

FIG. 4 shows plasma IL-2 concentration following intratracheal (IT) (400 μg/animal), subcutaneous (SC) or intravenous (IV) (0.5 mg/kg) administration of monomeric rhIL-2 with and without polysorbate 80 (Tween 80) or arginine in rats.

FIG. 5 shows plasma IL-2 concentration following intratracheal (IT) (400 μg/animal) administration of monomeric rhIL-2 and Proleukin with various surfactants (Poloxamer 188, PEG 4600, and polysorbate 80 (Tween 80) at various levels). All the monomeric rhIL-2 formulations contain arginine while the Proleukin formulations contain no arginine.

### DETAILED DESCRIPTION OF THE INVENTION

The present invention is directed to methods for administration of interleukin, more particularly interleukin-2 (IL-2) or variants thereof, to a subject via pulmonary inhalation. Interleukin-2 is a lymphokine that is produced by normal peripheral blood lymphocytes and is present in the body at low concentrations. It induces the proliferation of antigen- or mitogen-stimulated T cells after exposure to plant lectins, antigens, or other stimuli. IL-2 was first described by Morgan et al. (1976) Science 193:1007-1008 and originally called T cell growth factor because of its ability to induce proliferation of stimulated T lymphocytes. It is a protein with a reported molecular weight in the range of 13,000 to 17,000 (Gillis and Watson (1980) J. Exp. Med. 159:1709) and has an isoelectric point in the range of 6-8.5. It is now recognized that in addition to its growth factor properties, it modulates various in vitro and in vivo functions of the immune system. IL-2 is one of several lymphocyte-produced messenger-regulatory molecules that mediate cellular interactions and functions. The methods described herein encompass pulmonary inhalation of IL-2 or variants thereof. IL-2 proteins and variants thereof encompassed by the present invention are disclosed in detail below, following disclosure of the methods and compositions of the invention.

The methods of the invention comprise preparing a pharmaceutical composition comprising IL-2 or variants thereof in a form that is suitable for pulmonary delivery and administering the preparation to the subject via pulmonary inhalation. By “pulmonary inhalation” is intended the pharmaceutical composition is directly administered to the lung by delivering the composition in an aerosol or other suitable preparation from a delivery device into the oral cavity of the subject as the subject inhales through the oral cavity. By “aerosol” is intended a suspension of solid or liquid particles in flowing air or other physiologically acceptable gas stream. Other suitable preparations include, but are not limited to, mist, vapor, or spray preparations so long as the particles comprising the protein composition are delivered in a size range consistent with that described for a dry powder form of the pharmaceutical composition as defined below. Pulmonary inhalation could also be accomplished by other suitable methods known to those skilled in the art. These may include liquid instillation using a suitable device or other such methods. Pulmonary inhalation results in deposition of the inhaled protein composition in the alveoli of the subject's lungs. Once deposited, the protein may be absorbed, passively or actively, across the alveoli epithelium and capillary epithelium layers into the bloodstream for subsequent systemic distribution.

Pulmonary administration of a polypeptide or protein such as IL-2 requires dispensing of the biologically active substance from a delivery device into the oral cavity of a subject during inhalation. For purposes of the present invention, compositions comprising IL-2 or variants thereof are administered via inhalation of an aerosol or other suitable preparation that is obtained from an aqueous or nonaqueous solution or suspension form, or a solid or dry powder form of the pharmaceutical composition, depending upon the delivery device used. Such delivery devices are well known in the art and include, but are not limited to, nebulizers, metered-dose inhalers, and dry powder inhalers, or any other appropriate delivery mechanisms that allow for dispensing of a pharmaceutical composition as an aqueous or nonaqueous solution or suspension or as a solid or dry powder form. By “aqueous” is intended a composition prepared with, containing, or dissolved in water, including mixtures wherein water is the predominating substance in the mixture. A predominating substance is present in a greater quantity than another component of the mixture. By “nonaqueous” is intended a composition prepared with, containing, or dissolved in a substance other than water or mixtures wherein water is not the predominating substance in the mixture. By “solution” is intended a homogeneous preparation of two or more substances, which may be solids, liquids, gases, or intercombinations thereof. By “suspension” is intended a mixture of substances such that one or more insoluble substances are homogeneously dispersed in another predominating substance.

For purposes of the present invention, the terms “solid” and “dry powder” are used interchangeably. By “solid” or “dry powder” form of a pharmaceutical composition is intended the composition has been dried to a finely divided powder having a moisture content below about 10% by weight, usually below about 5% by weight, and preferably below about 3% by weight. This dry powder form of the composition consists of particles comprising the IL-2 or variants thereof. Preferred particle sizes are less than about 10.0 μm mean diameter, more preferably less than about 7.0 μm, even more preferably about less than about 6.0 μm, even more preferably in the range of 0.1 to 5.0 μm, most preferably in the range of about 1.0 to about 5.0 μm mean diameter.

Thus, a liquid pharmaceutical composition comprising IL-2 or variants thereof intended for use in the methods of the present invention may either be used as a liquid solution or suspension in the delivery device or first be processed into a dry powder form using lyophilization or spray-drying techniques well known in the art. Where a liquid solution or suspension is used in the delivery device, a nebulizer, a metered dose inhaler, or other suitable delivery device delivers, in a single or multiple fractional dose, by pulmonary inhalation a pharmaceutically effective amount of the composition to the subject's lungs as droplets having the same particle size range noted above for the dry powder form. By “pharmaceutically effective amount” is intended an amount that is useful in the treatment, prevention, or diagnosis of a disease or condition. The liquid solution or suspension of the composition may be used with physiologically appropriate stabilizing agents, excipients, bulking agents, surfactants, or combinations thereof, as discussed below. Examples of suitable excipients include, but are not limited to, buffers, viscosity modifiers, or other therapeutically inactive but functional additives.

Where the liquid pharmaceutical composition is lyophilized prior to use in the delivery methods of the invention, the lyophilized composition is milled to obtain the finely divided dry powder consisting of particles within the desired size range noted above. Where spray-drying is used to obtain a dry powder form of the liquid pharmaceutical composition, the process is carried out under conditions that result in a substantially amorphous finely divided dry powder consisting of particles within the desired size range noted above. Similarly, if the starting pharmaceutical composition is already in a lyophilized form, the composition can be milled to obtain the dry powder form for subsequent preparation as an aerosol or other preparation suitable for pulmonary inhalation. Where the starting pharmaceutical composition is in its spray-dried form, the composition has preferably been prepared such that it is already in a dry powder form having the appropriate particle size for dispensing as an aqueous or nonaqueous solution or suspension or dry powder form in accordance with the pulmonary administration methods of the invention. For methods of preparing dry powder forms of pharmaceutical compositions, see, for example, WO 96/32149, WO 97/41833, WO 98/29096, and U.S. Pat. Nos. 5,976,574, 5,985,248, and 6,001,336; herein incorporated by reference.

The resulting dry powder form of the composition is then placed within an appropriate delivery device for subsequent preparation as an aerosol or other suitable preparation that is delivered to the subject via pulmonary inhalation. Where the dry powder form of the pharmaceutical composition is to be prepared and dispensed as an aqueous or nonaqueous solution or suspension, a metered-dose inhaler, or other appropriate delivery device is used. A pharmaceutically effective amount of the dry powder form of the composition is administered in an aerosol or other preparation suitable for pulmonary inhalation. The amount of dry powder form of the composition placed within the delivery device is sufficient to allow for delivery of a pharmaceutically effective amount of the composition to the subject by inhalation. Thus, the amount of dry powder form to be placed in the delivery device will compensate for possible losses to the device during storage and delivery of the dry powder form of the composition. Following placement of the dry powder form within a delivery device, the properly sized particles as noted above are suspended in an aerosol propellant. The pressurized nonaqueous suspension is then released from the delivery device into the air passage of the subject while inhaling. The delivery device delivers, in a single or multiple fractional dose, by pulmonary inhalation a pharmaceutically effective amount of the composition to the subject's lungs. The aerosol propellant may be any conventional material employed for this purpose, such as a chlorofluorocarbon, a hydrochloro-fluorocarbon, a hydrofluorocarbon, or a hydrocarbon, including trichlorofluoromethane, dichlorodifluro-methane, dichlorotetrafluoromethane, dichlorodifluoro-methane, dichlorotetrafluoroethanol, and 1,1,1,2-tetra-fluoroethane, or combinations thereof. A surfactant may be added to the pharmaceutical composition to reduce adhesion of the protein-containing dry powder to the walls of the delivery device from which the aerosol is dispensed. Suitable surfactants for this intended use include, but are not limited to, sorbitan trioleate, soya lecithin, and oleic acid. Devices suitable for pulmonary delivery of a dry powder form of a protein composition as a nonaqueous suspension are commercially available. Examples of such devices include the Ventolin metered-dose inhaler (Glaxo Inc., Research Triangle Park, N.C.) and the Intal Inhaler (Fisons, Corp., Bedford, Mass.). See also the aerosol delivery devices described in U.S. Pat. Nos. 5,522,378, 5,775,320, 5,934,272 and 5,960,792, herein incorporated by reference.

Where the solid or dry powder form of the pharmaceutical composition is to be delivered as a dry powder form, a dry powder inhaler or other appropriate delivery device is preferably used. The dry powder form of the pharmaceutical composition is preferably prepared as a dry powder aerosol by dispersion in a flowing air or other physiologically acceptable gas stream in a conventional manner. Examples of commercially available dry powder inhalers suitable for use in accordance with the methods herein include the Spinhaler powder inhaler (Fisons Corp., Bedford, Mass.) and the Ventolin Rotahaler (Glaxo, Inc., Research Triangle Park, N.C.). See also the dry powder delivery devices described in WO 93/00951, WO 96/09085, WO 96/32152, and U.S. Pat. Nos. 5,458,135, 5,785,049, and 5,993,783, herein incorporated by reference.

The dry powder form of the pharmaceutical composition comprising IL-2 or variants thereof can be reconstituted to an aqueous solution for subsequent delivery as an aqueous solution aerosol using a nebulizer, a metered dose inhaler, or other suitable delivery device. In the case of a nebulizer, the aqueous solution held within a fluid reservoir is converted into an aqueous spray, only a small portion of which leaves the nebulizer for delivery to the subject at any given time. The remaining spray drains back into a fluid reservoir within the nebulizer, where it is aerosolized again into an aqueous spray. This process is repeated until the fluid reservoir is completely dispensed or until administration of the aerosolized spray is terminated. Such nebulizers are commercially available and include, for example, the Ultravent nebulizer (Mallinckrodt Inc., St. Louis, Mo.) and the Acorn II nebulizer (Marquest Medical Products, Englewood, Colo.). See also the nebulizer described in WO 93/00951, and the device for delivering aerosolized aqueous formulations described in U.S. Pat. No. 5,544,646; herein incorporated by reference.

Any IL-2 pharmaceutical composition can be used in the methods of the invention. Such pharmaceutical compositions are known in the art and include, but are not limited to, those disclosed in U.S. Pat. Nos. 4,745,180; 4,766,106; 4,816,440; 4,894,226; 4,931,544; and 5,078,997, all of which are herein incorporated by reference. Thus liquid, lyophilized, or spray-dried compositions comprising IL-2 or variants thereof and which are known in the art may be prepared as an aqueous or nonaqueous solution or suspension or as a dry powder form for subsequent administration to a subject via pulmonary inhalation. Each of these compositions will comprise IL-2 or variants thereof as a therapeutically or prophylactically active component. By “therapeutically or prophylactically active component” is intended the IL-2 or variants thereof is specifically incorporated into the composition to bring about a desired therapeutic or prophylactic response with regard to treatment, prevention, or diagnosis of a disease or condition within a subject when the pharmaceutical composition is administered to that subject. Preferably the pharmaceutical compositions comprise appropriate stabilizing agents, bulking agents, or both to minimize problems associated with loss of protein stability and biological activity during lyophilization, spray-drying, and aerosolizing processes included in the pulmonary administration methods of the invention.

In preferred embodiments of the invention, the pharmaceutical compositions useful in the methods of the invention are compositions comprising stabilized monomeric IL-2 or variants thereof, compositions comprising multimeric IL-2 or variants thereof, compositions comprising stabilized lyophilized or spray-dried IL-2 or variants thereof, and highly absorbable forms of these compositions as noted herein.

Pharmaceutical compositions comprising stabilized monomeric IL-2 or variants thereof are disclosed in the copending provisional application entitled “Stabilized Liquid Polypeptide-Containing Pharmaceutical Compositions,” filed Oct. 4, 1999 and assigned U.S. Provisional application Ser. No. 60/157,696, the disclosure of which is herein incorporated by reference. By “monomeric” IL-2 is intended the protein molecules are present substantially in their monomer form, not in an aggregated form, in the pharmaceutical compositions described herein. Hence covalent or hydrophobic oligomers or aggregates of IL-2 are not present. Briefly, the IL-2 or variants thereof in these liquid compositions is formulated with an amount of an amino acid base sufficient to decrease aggregate formation of IL-2 or variants thereof during storage. The amino acid base is an amino acid or a combination of amino acids, where any given amino acid is present either in its free base form or in its salt form. Preferred amino acids are selected from the group consisting of arginine, lysine, aspartic acid, and glutamic acid. These compositions further comprise a buffering agent to maintain pH of the liquid compositions within an acceptable range for stability of IL-2 or variants thereof, where the buffering agent is an acid substantially free of its salt form, an acid in its salt form, or a mixture of an acid and its salt form. Preferably the acid is selected from the group consisting of succinic acid, citric acid, phosphoric acid, and glutamic acid.

The amino acid base in these compositions serves to stabilize the IL-2 or variants thereof against aggregate formation during storage of the liquid pharmaceutical composition, while use of an acid substantially free of its salt form, an acid in its salt form, or a mixture of an acid and its salt form as the buffering agent results in a liquid composition having an osmolarity that is nearly isotonic. The liquid pharmaceutical composition may additionally incorporate other stabilizing agents, more particularly methionine, a nonionic surfactant such as polysorbate 80, and EDTA, to further increase stability of the polypeptide. Such liquid pharmaceutical compositions are said to be stabilized, as addition of amino acid base in combination with an acid substantially free of its salt form, an acid in its salt form, or a mixture of an acid and its salt form, results in the compositions having increased storage stability relative to liquid pharmaceutical compositions formulated in the absence of the combination of these two components.

These liquid pharmaceutical compositions comprising stabilized monomeric IL-2 or variants thereof may either be used in an aqueous form or prepared as a solid or dry powder form for use in the methods of the present invention, as noted above.

Examples of pharmaceutical compositions comprising multimeric human IL-2 or variants thereof are disclosed in commonly owned U.S. Pat. No. 4,604,377, the disclosure of which is herein incorporated by reference. By “multimeric” is intended the protein molecules are present in the pharmaceutical composition in a microaggregated form having an average molecular association of 10-50 molecules. These multimers are present as loosely bound, physically-associated IL-2 molecules. A lyophilized form of these compositions is available commercially under the tradename Proleukin (Chiron Corporation). The lyophilized formulations disclosed in this reference comprise selectively oxidized, microbially produced recombinant human IL-2 (“rhIL-2”) in which the recombinant IL-2 is admixed with a water soluble carrier such as mannitol that provides bulk, and a sufficient amount of sodium dodecyl sulfate to ensure the solubility of the recombinant IL-2 in water. These compositions are suitable for reconstitution in aqueous injections for parenteral administration and are stable and well tolerated in human patients. When reconstituted, the IL-2 or variants thereof retains its multimeric state. Such lyophilized or liquid compositions comprising multimeric IL-2 or variants thereof are encompassed by the methods of the present invention.

When pharmaceutical compositions comprising IL-2 or variants thereof are processed into a solid or dry powder form for subsequent delivery as an aerosol, it may be desirable to have carrier materials present that serve as a bulking agent or stabilizing agent. In this manner, the present invention discloses stabilized lyophilized or spray-dried pharmaceutical compositions comprising IL-2 or variants thereof for use in the methods of the present invention. These compositions may further comprise at least one bulking agent, at least one agent in an amount sufficient to stabilize the protein during the drying process, or both. By “stabilized” is intended the IL-2 protein or variants thereof retains its monomeric or multimeric form as well as its other key properties of quality, purity, and potency following lyophilization or spray-drying to obtain the solid or dry powder form of the composition.

Preferred carrier materials for use as a bulking agent include glycine, mannitol, alanine, valine, or any combination thereof, most preferably glycine. The bulking agent is present in the formulation in the range of 0% to about 10% (w/v), depending upon the agent used. When the bulking agent is glycine, it is present in the range of about 0% to about 4%, preferably about 0.25% to about 3.5%, more preferably about 0.5% to 3.0%, even more preferably about 1.0% to about 2.5%, most preferably about 2.0%. When the bulking agent is mannitol, it is present in the range of about 0% to about 5.0%, preferably about 1.0% to about 4.5%, more preferably about 2.0% to about 4.0%, most preferably about 4.0%. When the bulking agent is alanine or valine, it is present in the range of about 0% to about 5.0%, preferably about 1.0% to about 4.0%, more preferably about 1.5% to about 3.0%, most preferably about 2.0%.

Preferred carrier materials for use as a stabilizing agent include any sugar or sugar alcohol or any amino acid. Preferred sugars include sucrose, trehalose, raffinose, stachyose, sorbitol, glucose, lactose, dextrose or any combination thereof, preferably sucrose. When the stabilizing agent is a sugar, it is present in the range of about 0% to about 9.0% (w/v), preferably about 0.5% to about 5.0%, more preferably about 1.0% to about 3.0%, most preferably about 1.0%. When the stabilizing agent is an amino acid, it is present in the range of about 0% to about 1.0% (w/v), preferably about 0.3% to about 0.7%, most preferably about 0.5%.

These stabilized lyophilized or spray-dried compositions may optionally comprise methionine, ethylenediaminetetracetic acid (EDTA) or one of its salts such as disodium EDTA or other chelating agent, which protect the IL-2 or variants thereof against methionine oxidation. Use of these agents in this manner is described in copending U.S. Provisional application Serial No. 60/157696, herein incorporated by reference. Methionine is present in the stabilized lyophilized or spray-dried pharmaceutical compositions at a concentration of about 0 to about 10.0 mM, preferably about 1.0 to about 9.0 mM, more preferably about 2.0 to about 8.0 mM, even more preferably about 3.0 to about 7.0 mM, still more preferably about 4.0 to about 6.0 mM, most preferably about 5.0 mM. EDTA is present at a concentration of about 0 to about 10.0 mM, preferably about 0.2 mM to about 8.0 mM, more preferably about 0.5 mM to about 6.0 mM, even more preferably about 0.7 mM to about 4.0 mM, still more preferably about 0.8 mM to about 3.0 mM, even more preferably about 0.9 mM to about 2.0 mM, most preferably about 1.0 mM.

The stabilized lyophilized or spray-dried compositions may be formulated using a buffering agent, which maintains the pH of the pharmaceutical composition within an acceptable range when in a liquid phase, such as during the formulation process or following reconstitution of the dried form of the composition. Preferably the pH is in the range of about pH 4.0 to about pH 8.5, more preferably about pH 4.5 to about pH 7.5, even more preferably about pH 5.0 to about pH 6.5, more preferably still about pH 5.6 to about pH 6.3, and most preferably about pH 5.7 to about pH 6.2. Suitable pH's include about 4.0, about 4.5, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, up to about 8.5. Most preferably, the pH is about 5.8.

Suitable buffering agents include, but are not limited to, citrate buffer, phosphate buffer, succinate buffer, more particularly a sodium citrate/citric acid. Alternatively imidazole or histidine or other base/acid that maintains pH in the range of about pH 4.0 to about 8.5 can be used. Buffers are chosen such that they are compatible with the drying process and do not affect the quality, purity, potency, and stability of the protein during processing and upon storage.

In one embodiment of the invention, the stabilized lyophilized or spray-dried pharmaceutical compositions comprise IL-2 or variants thereof, glycine in the range of about 0% to about 2.0%, sucrose in the range of about 0% to about 9.0%, methionine at a concentration of about 0 mM to about 10.0 mM, and EDTA at about 0 mM to about 10.0 mM buffered to a pH of about pH 5.0 to about pH 8.0 with a 10.0 mM sodium citrate/citric acid buffer. In a preferred embodiment the stabilized lyophilized or spray-dried pharmaceutical composition comprising IL-2 or variants thereof further comprises 2.0% glycine, 1.0% sucrose, 5.0 mM methionine, 1.0 mM EDTA, and greater than 0% to about 1.0% polysorbate 80, buffered to a pH of about pH 6.0 to about pH 7.0 with a 10.0 mM sodium citrate/citric acid buffer. The concentration of IL-2 or variants thereof in these compositions is about 0.01 mg/ml to about 1.0 mg/ml, preferably about 0.2 mg/ml to about 0.8 mg/ml, more preferably about 0.3 mg/ml to about 0.6 mg/ml, most preferably about 0.3 mg/ml to about 0.5 mg/ml.

Any of the pharmaceutical compositions comprising IL-2 or variants thereof contemplated for use in the methods of the invention may be formulated with at least one surfactant in an amount sufficient to enhance absorption of the inhaled particles comprising IL-2 or variants thereof to obtain a highly absorbable composition for use in pulmonary inhalation in accordance with the methods described herein. By “highly absorbable” is intended the pharmaceutical compositions comprising IL-2 or variants thereof and at least one surfactant, when used in the pulmonary administration methods of the present invention, have a bioavailability that is about 1.5- to about 20-fold, preferably about 1.6- to about 17-fold, more preferably about 1.7- to about 15-fold, even more preferably about 1.8- to about 13-fold, still more preferably about 1.9- to about 11-fold, most preferably about 2.0- to about 10-fold greater than the comparable formulation comprising IL-2 or variants thereof in the absence of the surfactant. A surfactant used in this manner results in enhanced bioavailability of the inhaled IL-2 or variants thereof as disclosed below.

Any surfactant that enhances absorption of a pharmaceutical composition comprising IL-2 or variants thereof in the manner disclosed herein may be used to obtain these highly absorbable protein-containing pharmaceutical compositions. Surfactants suitable for use in enhancing absorption of the inhaled IL-2 or variants thereof include, but are not limited to, polyoxyethylene sorbitol esters such as polysorbate 80 (Tween 80) and polysorbate 20 (Tween 20); polyoxypropylene-polyoxyethylene esters such as Poloxamer 188; polyoxyethylene alcohols such as Brij 35; a mixture of polysorbate surfactants with phospholipids such as phosphatidylcholine and derivatives (dipalmitoyl, dioleoyl, dimyristyl, or mixed derivatives such as 1-palmitoyl, 2-olcoyl, etc.), dimyristolglycerol and other members of the phospholipid glycerol series; lysophosphatidylcholine and derivatives thereof; mixtures of polysorbates with lysolecithin or cholesterol; a mixture of polysorbate surfactants with sorbitan surfactants (such as sorbitan monoleate, dioleate, trioleate or others from this class); poloxamer surfactants; bile salts and their derivatives such as sodium cholate, sodium deoxycholate, sodium glycodeoxycholate, sodium taurocholate, etc.; mixed micelles of IL-2 with bile salts and phospholipids; Brij surfactants (such as Brij 35-PEG923) lauryl alcohol, etc.). The amount of surfactant to be added is in the range of about 0.005% to about 1.0% (w/v), preferably about 0.005% to about 0.5%, more preferably about 0.01% to about 0.4%, even more preferably about 0.03% to about 0.3%, most preferably about 0.05% to about 0.2%.

In one embodiment, the highly absorbable pharmaceutical composition comprises monomeric IL-2 with at least one surfactant as noted above in an amount sufficient to enhance absorption of the inhaled IL-2. This composition is similar to the highly absorbable pharmaceutical composition comprising stabilized monomeric IL-2 or variants thereof, but without the amino acid base present.

The foregoing optional stabilizing or bulking agents and surfactants may be added to a pharmaceutical composition prior to preparing the composition as a solid or dry powder form. In this case, these agents are formed simultaneously with and as part of the particles comprising IL-2 or variants thereof. When prepared in this manner, the IL-2 or variants thereof is present in each individual particle at a weight percent in the range from about 0.01% to about 100%, preferably from about 0.1% to about 10%. The remainder of the particle is primarily the stabilizing agents but also includes buffer and other components as noted above. Alternatively, any of these agents not already present in the pharmaceutical composition may be prepared separately in a dry powder form and then combined with the dry powder form of the pharmaceutical composition prior to preparing the final dry powder form of the composition as an aerosol.

In accordance with the method of the present invention, the aqueous or nonaqueous solution or suspension or solid or dry powder form of the composition comprising IL-2 or variants thereof is administered to a subject in the form of an aerosol or other preparation suitable for pulmonary inhalation. By “subject” is intended any animal. Preferably the subject is mammalian, most preferably the subject is human. Mammals of particular importance other than human include, but are not limited to, dogs, cats, cows, horses, sheep, and pigs.

When administration is for the purpose of treatment, administration may be for either a prophylactic or therapeutic purpose. When provided prophylactically, the substance is provided in advance of any symptom. The prophylactic administration of the substance serves to prevent or attenuate any subsequent symptom. When provided therapeutically, the substance is provided at (or shortly after) the onset of a symptom. The therapeutic administration of the substance serves to attenuate any actual symptom.

The present invention also provides a method for enhancing bioavailability of IL-2 or variants thereof administered by pulmonary inhalation. The method comprises preparing the highly absorbable compositions described herein as an aerosol or other suitable preparation and administering the aerosol or other suitable preparation to the subject via pulmonary inhalation. By “bioavailability” is intended a measure of the absorption of IL-2 or variants thereof by the lung tissues into the blood stream following pulmonary administration of IL-2 or variants thereof when compared to the amount of IL-2 or variants thereof in the bloodstream following intravenous injection (absolute bioavailability) or subcutaneous injection (relative bioavailability). Bioavailability of the inhaled IL-2 or variants thereof may be enhanced with the addition of a surfactant to a pharmaceutical composition comprising IL-2 or variants thereof prior to its administration to the subject.

For purposes of the present invention, bioavailability of an IL-2 containing pharmaceutical composition is determined using an intratracheal (IT) technique (Niven et al. (1994) Pharm. Res. 12:1142-1149; Niven et al. (1995) Pharm. Res. 12:1889-1895). In this manner, a solution comprising an IL-2 formulation is injected from a syringe into a mammal either directly into the trachea (IT), directly into a vein (IV), or subcutaneously (SC). Blood is then collected at intervals and analyzed for presence of IL-2. Bioavailability is then estimated as the dose-normalized area under the curve (AUC) ratio after IT (or SC) versus IV administration (absolute bioavailability) or IT versus SC administration (relative bioavailability), the details of which are described in the examples below.

The IL-2 present in the pharmaceutical compositions described herein for use in the methods of the invention may be native or obtained by recombinant techniques, and may be from any source, including mammalian sources such as, e.g., mouse, rat, rabbit, primate, pig, and human. Preferably such polypeptides are derived from a human source, and more preferably are recombinant, human proteins from microbial hosts.

The pharmaceutical compositions useful in the methods of the invention may comprise biologically active variants of IL-2. Such variants should retain the desired biological activity of the native polypeptide such that the pharmaceutical composition comprising the variant polypeptide has the same therapeutic effect as the pharmaceutical composition comprising the native polypeptide when administered to a subject. That is, the variant polypeptide will serve as a therapeutically active component in the pharmaceutical composition in a manner similar to that observed for the native polypeptide. Methods are available in the art for determining whether a variant polypeptide retains the desired biological activity, and hence serves as a therapeutically active component in the pharmaceutical composition. Biological activity can be measured using assays specifically designed for measuring activity of the native polypeptide or protein, including assays described in the present invention. Additionally, antibodies raised against a biologically active native polypeptide can be tested for their ability to bind to the variant polypeptide, where effective binding is indicative of a polypeptide having a conformation similar to that of the native polypeptide.

Suitable biologically active variants of native or naturally occurring IL-2 can be fragments, analogues, and derivatives of that polypeptide. By “fragment” is intended a polypeptide consisting of only a part of the intact polypeptide sequence and structure, and can be a C-terminal deletion or N-terminal deletion of the native polypeptide. By “analogue” is intended an analogue of either the native polypeptide or of a fragment of the native polypeptide, where the analogue comprises a native polypeptide sequence and structure having one or more amino acid substitutions, insertions, or deletions. “Muteins”, such as those described herein, and peptides having one or more peptoids (peptide mimics) are also encompassed by the term analogue (see International Publication No. WO 91/04282). By “derivative” is intended any suitable modification of the native polypeptide of interest, of a fragment of the native polypeptide, or of their respective analogues, such as glycosylation, phosphorylation, polymer conjugation (such as with polyethylene glycol), or other addition of foreign moieties, so long as the desired biological activity of the native polypeptide is retained. Methods for making polypeptide fragments, analogues, and derivatives are generally available in the art.

For example, amino acid sequence variants of the polypeptide can be prepared by mutations in the cloned DNA sequence encoding the native polypeptide of interest. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (1989) Molecular Cloning: A Laboratory Manual (Cold Spring Harbor, N.Y.); U.S. Pat. No. 4,873,192; and the references cited therein; herein incorporated by reference. Guidance as to appropriate amino acid substitutions that do not affect biological activity of the polypeptide of interest may be found in the model of Dayhoff et al. (1978) in Atlas of Protein Sequence and Structure (Natl. Biomed. Res. Found., Washington, D.C.), herein incorporated by reference. Conservative substitutions, such as exchanging one amino acid with another having similar properties, may be preferred. Examples of conservative substitutions include, but are not limited to, Gly [symbol] Ala, Val [symbol] Ile [symbol] Leu, Asp [symbol] Glu, Lys [symbol] Arg, Asn [symbol] Gln, and Phe [symbol] Trp [symbol] Tyr.

In constructing variants of the IL-2 polypeptide of interest, modifications are made such that variants continue to possess the desired activity. Obviously, any mutations made in the DNA encoding the variant polypeptide must not place the sequence out of reading frame and preferably will not create complementary regions that could produce secondary mRNA structure. See EP Patent Application Publication No. 75,444.

Biologically active variants of IL-2 will generally have at least 70%, preferably at least 80%, more preferably about 90% to 95% or more, and most preferably about 98% or more amino acid sequence identity to the amino acid sequence of the reference polypeptide molecule, which serves as the basis for comparison. A biologically active variant of a native polypeptide of interest may differ from the native polypeptide by as few as 1-15 amino acids, as few as 1-10, such as 6-10, as few as 5, as few as 4, 3, 2, or even 1 amino acid residue. By “sequence identity” is intended the same amino acid residues are found within the variant polypeptide and the polypeptide molecule that serves as a reference when a specified, contiguous segment of the amino acid sequence of the variants is aligned and compared to the amino acid sequence of the reference molecule. The percentage sequence identity between two amino acid sequences is calculated by determining the number of positions at which the identical amino acid residue occurs in both sequences to yield the number of matched positions, dividing the number of matched positions by the total number of positions in the segment undergoing comparison to the reference molecule, and multiplying the result by 100 to yield the percentage of sequence identity.

For purposes of optimal alignment of the two sequences, the contiguous segment of the amino acid sequence of the variants may have additional amino acid residues or deleted amino acid residues with respect to the amino acid sequence of the reference molecule. The contiguous segment used for comparison to the reference amino acid sequence will comprise at least twenty (20) contiguous amino acid residues, and may be 30, 40, 50, 100, or more residues. Corrections for increased sequence identity associated with inclusion of gaps in the variants' amino acid sequence can be made by assigning gap penalties. Methods of sequence alignment are well known in the art for both amino acid sequences and for the nucleotide sequences encoding amino acid sequences.

Thus, the determination of percent identity between any two sequences can be accomplished using a mathematical algorithm. One preferred, non-limiting example of a mathematical algorithm utilized for the comparison of sequences is the algorithm of Myers and Miller (1988) CABIOS 4:11-17. Such an algorithm is utilized in the ALIGN program (version 2.0), which is part of the GCG sequence alignment software package. A PAM120 weight residue table, a gap length penalty of 12, and a gap penalty of 4 can be used with the ALIGN program when comparing amino acid sequences. Another preferred, nonlimiting example of a mathematical algorithm for use in comparing two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences homologous to a nucleotide sequence encoding the polypeptide of interest. BLAST protein searches can be performed with the XBLAST program, score=50, wordlength=3, to obtain amino acid sequences homologous to the polypeptide of interest. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. See http://www.ncbi.nlm.nih.gov. Also see the ALIGN program (Dayhoff (1978) in Atlas of Protein Sequence and Structure 5:Suppl. 3 (National Biomedical Research Foundation, Washington, D.C.)) and programs in the Wisconsin Sequence Analysis Package, Version 8 (available from Genetics Computer Group, Madison, Wis.), for example, the GAP program, where default parameters of the programs are utilized.

When considering percentage of amino acid sequence identity, some amino acid residue positions may differ as a result of conservative amino acid substitutions, which do not affect properties of protein function. In these instances, percent sequence identity may be adjusted upwards to account for the similarity in conservatively substituted amino acids. Such adjustments are well known in the art. See, for example, Myers and Miller (1988) Computer Applic. Biol. Sci. 4:11-17.

The precise chemical structure of a polypeptide depends on a number of factors. As ionizable amino and carboxyl groups are present in the molecule, a particular polypeptide may be obtained as an acidic or basic salt, or in neutral form. All such preparations that retain their biological activity when placed in suitable environmental conditions are included in the definition of polypeptides as used herein. Further, the primary amino acid sequence of the polypeptide may be augmented by derivatization using sugar moieties (glycosylation) or by other supplementary molecules such as lipids, phosphate, acetyl groups and the like. It may also be augmented by conjugation with saccharides. Certain aspects of such augmentation are accomplished through post-transnational processing systems of the producing host; other such modifications may be introduced in vitro. In any event, such modifications are included in the definition of polypeptide used herein so long as the activity of the polypeptide is not destroyed. It is expected that such modifications may quantitatively or qualitatively affect the activity, either by enhancing or diminishing the activity of the polypeptide, in the various assays. Further, individual amino acid residues in the chain may be modified by oxidation, reduction, or other derivatization, and the polypeptide may be cleaved to obtain fragments that retain activity. Such alterations that do not destroy activity do not remove the polypeptide sequence from the definition of polypeptide of interest as used herein.

The art provides substantial guidance regarding the preparation and use of polypeptide variants. In preparing the IL-2 variants, one of skill in the art can readily determine which modifications to the native protein nucleotide or amino acid sequence will result in a variant that is suitable for use as a therapeutically active component of a pharmaceutical composition used in the methods of the present invention.

The IL-2 or variants thereof for use in the methods and compositions of the present invention may be from any source, but preferably is recombinant IL-2. By “recombinant IL-2” is intended interleukin-2 having comparable biological activity to native-sequence IL-2 and which has been prepared by recombinant DNA techniques as described, for example, by Taniguchi et al. (1983) Nature 302:305-310 and Devos (1983) Nucleic Acids Research 11:4307-4323 or mutationally altered IL-2 as described by Wang et al. (1984) Science 224:1431-1433. In general, the gene coding for IL-2 is cloned and then expressed in transformed organisms, preferably a microorganism, and most preferably E. coli, as described herein. The host organism expresses the foreign gene to produce IL-2 under expression conditions. Synthetic recombinant IL-2 can also be made in eukaryotes, such as yeast or human cells. Processes for growing, harvesting, disrupting, or extracting the IL-2 from cells are substantially described in, for example, U.S. Pat. Nos. 4,604,377; 4,738,927; 4,656,132; 4,569,790; 4,748,234; 4,530,787; 4,572,798; 4,748,234; and 4,931,543, herein incorporated by reference in their entireties.

For examples of variant IL-2 proteins, see European Patent Application No. 136,489; European Patent Application No. 83101035.0 filed Feb. 3, 1983 (published Oct. 19, 1983 under Publication No. 91539); European Patent Application No. 82307036.2, filed Dec. 22, 1982 (published Sep. 14, 1983 under No. 88195); the recombinant IL-2 muteins described in European Patent Application No. 83306221.9, filed Oct. 13, 1983 (published May 30, 1984 under No. 109748), which is the equivalent to Belgian Patent No. 893,016, commonly owned U.S. Pat. No. 4,518,584; the muteins described in U.S. Pat. No. 4,752,585 and WO 99/60128; and the IL-2 mutein used in the examples herein and described in U.S. Pat. No. 4,931,543; all of which are herein incorporated by reference. Additionally, IL-2 can be modified with polyethylene glycol to provide enhanced solubility and an altered pharmokinetic profile (see U.S. Pat. No. 4,766,106, hereby incorporated by reference in its entirety).

The following examples are offered by way of illustration and not by way of limitation.

Experimental

IL-2 is a potent mitogen that stimulates T-cell proliferation. It has wide therapeutic application as a treatment for cancer metastasis, as an adjuvant for cancer therapy, and as a conjunctive agent for infectious diseases. With the progress of various clinical trials using IL-2 therapy, it has been realized that alternative, noninvasive administrative routes would be desirable.

Several pharmaceutical formulations comprising recombinant human IL-2 (“rhIL-2”) have been evaluated for their potential effectiveness in pulmonary administration. The formulations tested included Proleukin, a lyophilized rhIL-2 formulation currently marketed by Chiron Corp. for treating patients with metastatic renal cell carcinoma or metastatic melanoma. It is also in phase III trials for treating patients with HIV infection. It contains 0.05-2.0 mg/ml rhIL-2, 3-7% mannitol, 5.0-20.0 mM sodium phosphate, approximately 130-230 μg SDS/ml rhIL-2 at pH 5.5-8.0 after reconstitution with water-for-injection (WFI). The IL-2 in the formulation exists in a microaggregated form with an average molecular association of 10-50 molecules. The aggregation may affect lung absorption.

A newly developed stabilized liquid IL-2 formulation as disclosed in commonly owned U.S. Provisional application Serial No. 60/157696 has also been tested. The liquid IL-2 formulation is designated as monomeric rhIL2 in these studies. In contrast to the Proleukin formulation, the rhIL-2 molecule in this liquid formulation exists in a stabilized monomer form. This formulation contains 0.03-3.0 mg/ml rhIL-2, 150-300 mM L-arginine base, 50-150 mM succinic acid, 0.5-5.0 mM disodium EDTA, 1.0-10.0 mM methionine and 0.05-0.2% polysorbate 80 (Tween 80) at pH 5.0-8.0. It was hypothesized that the stabilized monomeric IL-2 formulation may be better absorbed through the deep lung surface than the multimeric Proleukin formulation. In addition, amino acids and polysorbate 80 used in the formulation may bring other advantages to the pulmonary delivery such as enhancement of IL-2 absorption across the lung tissue.

These IL-2 formulations have been evaluated for their relative pulmonary bioavailability as compared to subcutaneous injection using an intratracheal rat model. Sprague-Dawley rats were administered the two IL-2 formulations via either intratracheal instillation (IT) or subcutaneous (SC) injection. For each IT administration, the animals were first anesthetized using isoflurane or a CO₂/O₂ mixture and held in a sternal or upright position. All IT administered IL-2 doses were instilled with either a catheter or a ball-tipped needle using a 1-mL sterile disposable syringe. After IT dosing the animals were left upright for approximately 20 seconds to allow the dosing solution to settle into the lungs. Blood samples of these rats were collected at predetermined time intervals and heparinized. Plasma from the blood samples was separated and analyzed by immunoassay for IL-2 concentration.

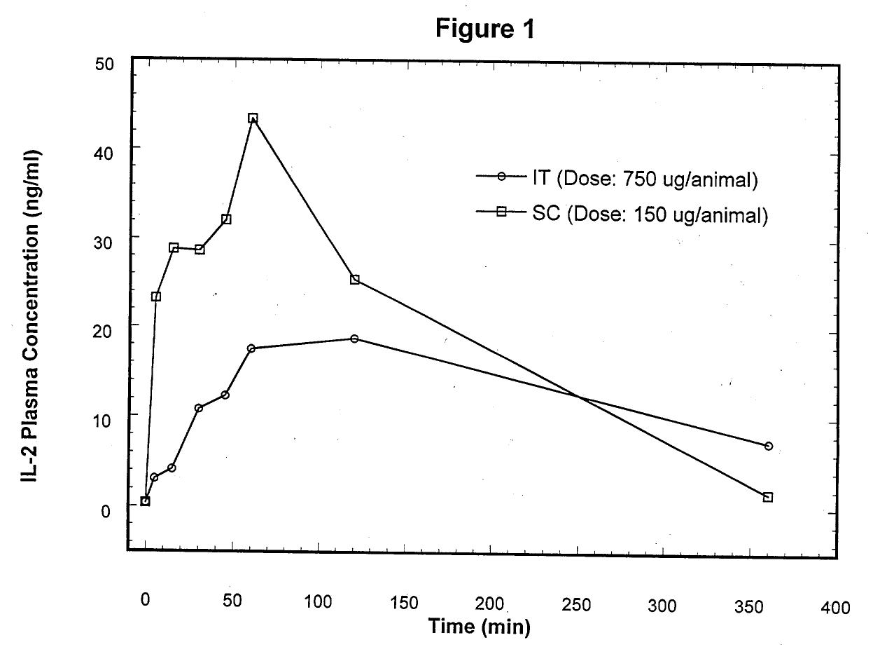

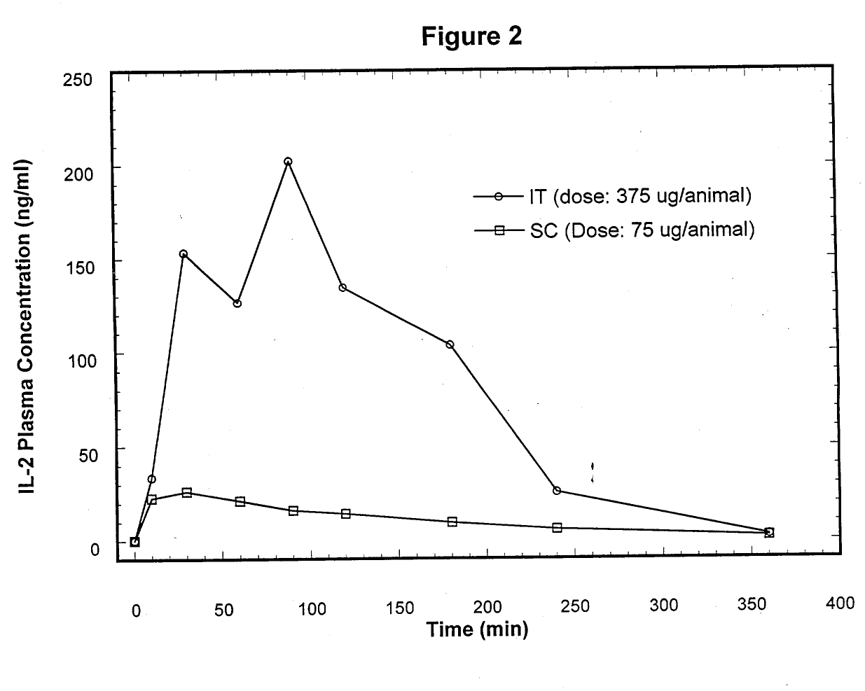

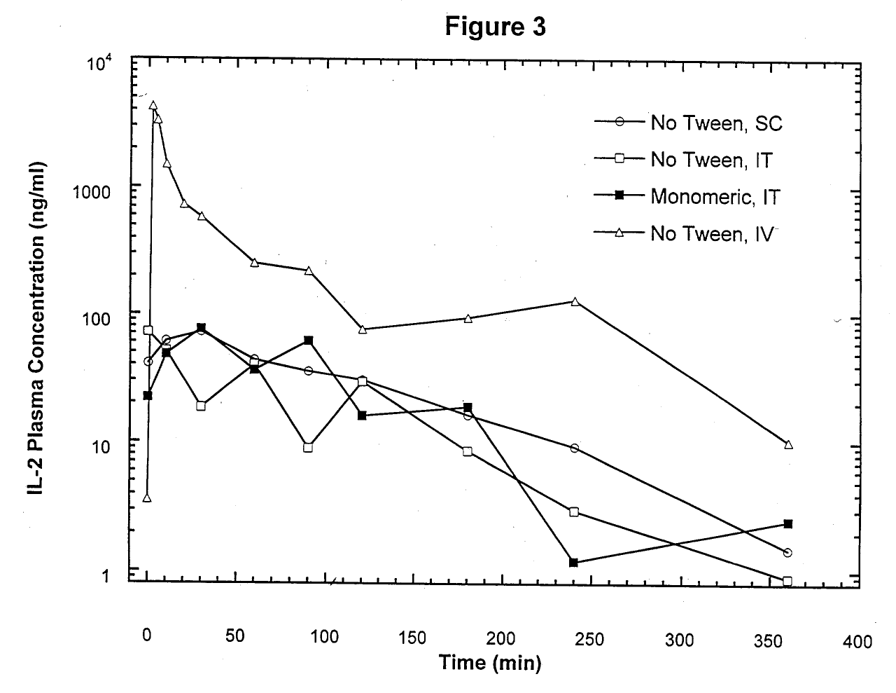

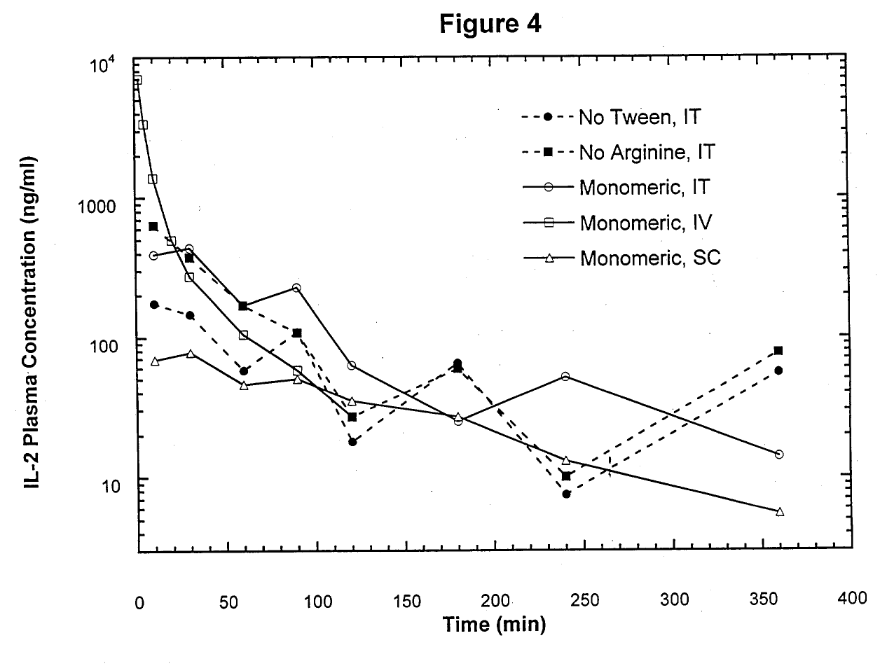

Four studies have been conducted to evaluate the systemic bioavailability of Proleukin and monomeric IL-2 formulations following intratracheal, subcutaneous, or intravenous delivery in rats. Monomeric formulations varied according to whether polysorbate 80 (Tween 80) and/or arginine was presented in the composition. Plasma IL-2 concentration profiles from the studies are shown in FIGS. 1-4. Pharmacokinetic parameters are summarized by study in Tables 1-4, respectively. The fraction of the dose absorbed systemically was calculated for each formulation as the dose-normalized AUC ratio after IT versus SC administration (relative bioavailability) or SC (or IT) versus IV administration (absolute bioavailability). For studies with no corresponding SC administration for some of the monomeric formulations, the relative bioavailability was calculated using AUC of SC delivery by a similar monomeric formulation based on previous experience that SC AUCs of these monomeric formulations were similar.

TABLE 1
PK parameters following IT or SC administration of the Proleukin
formulation in 8 rats (4/sex)^{a}.

| | Average | Dose per | | | | AUC | R.B. |
|---|---|---|---|---|---|---|---|
| | BW | animal | Cmax | Tmax | t_{1/2} | (0-inf.) | (IT/SC) |
| Route | (g) | (ug) | (ng/ml) | (hr) | (hr) | (ng-hr/ml) | (%) |
| IT | 319 | 750 | 19 | 2 | 3.7 | 80 | 14 |
| SC | 327 | 150 | 43 | 1 | 1.1 | 118 | |

^{a}The symbols used in the table:
IT for intratracheal administration,
SC for subcutaneous administration,
BW for body weight,
Cmax is the maximum plasma IL-2 concentration detected,
Tmax is the time for Cmax,
t_{1/2} is the half-life for clearance,
AUC is the area-under-the-IL-2 plasma vs. time curve, and
R.B. is the relative bioavailability calculated on a percentage basis by dividing AUC for IT by AUC for SC after dose normalization and then multiplying the resultant number by 100.

TABLE 2
PK parameters following IT or SC administration of the stabilized
monomeric rhIL-2 formulation (with polysorbate 80)
in 8 rats (4/sex)^{a}.

| | Average | Dose per | | | | AUC | R.B. |
|---|---|---|---|---|---|---|---|
| | BW | animal | Cmax | Tmax | t_{1/2} | (0-inf.) | (IT/SC) |
| Route | (g) | (ug) | (ng/ml) | (hr) | (hr) | (ng-hr/ml) | (%) |
| IT | 311 | 375 | 202 | 1.5 | 0.6 | 482 | 146 |
| SC | 320 | 75 | 26 | 0.5 | 1.3 | 66 | |

^{a}The symbols used in the table:
IT for intratracheal administration,
SC for subcutaneous administration,
BW for body weight,
Cmax is the maximum plasma IL-2 concentration detected,
Tmax is the time for Cmax,
t_{1/2} is the half-life for clearance,
AUC is the area-under-the-IL-2 plasma vs. time curve, and
R.B. is the relative bioavailability calculated on a percentage basis by dividing AUC for IT by AUC for SC after dose normalization and then multiplying the resultant number by 100.

TABLE 3
PK parameters following IT, SC or IV administration of the monomeric rhIL-2
formulation with or without polysorbate 80 (Tween 80) in 8 rats (4/sex)^{a}.

| | | Average | Dose per | | | AUC | R.B. | A.B. |
|---|---|---|---|---|---|---|---|---|
| | | BW | animal | Cmax | Tmax | (0-inf) | (IT/SC) | (SC/IV) |
| Tween | Route | (g) | (ug) | (ng/ml) | (hr) | (ng-hr/ml) | (%) | (%) |
| + | IT | ˜320 | 375 | 190 | 0.5 | 327 | 90^{b} | |
| − | IT | ˜320 | 375 | 51 | 0.2 | 88 | 24 | |
| − | SC | ˜320 | 150 | 72 | 0.5 | 145 | | 10 |
| − | LV | ˜320 | 150 | 4238 | 0 | 1532 | | |

^{a}The symbols used in the table:
IT for intratracheal administration,
SC for subcutaneous administration,
BW for body weight,
Cmax is the maximum plasma IL-2 concentration detected,
Tmax is the time for Cmax,
t_{1/2} is the half-life for clearance,
AUC is the area-under-the-IL-2 plasma vs. time curve,
R.B. is the relative bioavailability calculated by dividing AUC for IT by AUC for SC after dose normalization, and
A.B. is the absolute bioavailability calculated on a percentage basis by dividing AUC for SC by AUC for IV and then multiplying the resultant number by 100.
^{b}The relative bioavailability calculation of the monomeric formulation with polysorbate 80 uses AUC for SC of the monomeric formulation without polysorbate 80.

TABLE 4
PK parameters following IT, SC or IV administration of the
monomeric rhIL-2 formulation (with polysorbate 80 and arginine),
the monomeric formulation without polysorbate 80, and the
monomeric formulation without arginine in 4 male rats^{a}.

| | | Aver. | Dose per | | | AUC | R.B. | A.B. |
|---|---|---|---|---|---|---|---|---|
| | | BW | animal | Cmax | Tmax | (0-inf) | (IT/SC) | (SC/IV) |
| Formuation | Route | (g) | (ug) | (ng/ml) | (hr) | (ng-hr/ml) | (%) | (%) |
| Monomeric^{b} | IT | ˜400 | 400 | 441 | 0.5 | 697 | 185 | |
| Monomeric | SC | ˜400 | ˜200 | 156 | 0.5 | 188 | | 15 |
| Monomeric | IV | ˜400 | ˜200 | 14068 | 0 | 1250 | | |
| No Tween 80^{c} | IT | ˜400 | 400 | 176 | 0.17 | 298 | 79^{d} | |
| No arginine^{c} | IT | ˜400 | 400 | 637 | 0.17 | 530 | 141^{d} | |

^{a}The symbols used in the table:
IT for intratracheal administration,
SC for subcutaneous administration,
BW for body weight,
Cmax is the maximum plasma IL-2 concentration detected,
Tmax is the time for Cmax,
t_{1/2} is the half-life for clearance,
AUC is the area-under-the-IL-2 plasma vs. time curve,
R.B. is the relative bioavailability calculated by dividing AUC for IT by AUC for SC after dose normalization, and
A.B. is the absolute bioavailability calculated on a percentage basis by dividing AUC for SC by AUC for IV and then multiplying the resultant number by 100.
^{b}Formulation contained 0.1% polysorbate 80 and 230 mM arginine.
^{c}Formulation contained 230 mM arginine.
^{d}The relative bioavailability calculation of the monomeric formulations without polysorbate 80 and without arginine uses AUC for SC of the monomeric formulation.
^{e}Formulation contained 0.1% polysorbate 80.

The above data regarding systemic absorption of IL-2 via these administration routes can be summarized as follows. The relative bioavailability of Proleukin after IT dosing compared to SC dosing was 14% (Table 1). Monomeric IL-2 containing polysorbate 80 was absorbed significantly better than Proleukin after IT delivery. The absolute bioavailability of SC to IV was 15% (Table 4), while the relative bioavailability of IT to SC averaged 140% (Tables 2, 3, 4), indicating that IT dosing was absorbed equally well or better than SC dosing. Monomeric IL-2 containing polysorbate 80 but no arginine was absorbed about as well as monomeric IL-2 containing both polysorbate 80 and arginine (Table 4). Monomeric IL-2 without polysorbate 80 was absorbed better than Proleukin but not as well as monomeric IL-2 containing polysorbate 80. The absolute bioavailability was 10% (Table 3) and the relative bioavailability was 52% (Table 3 and 4).

A fifth study was conducted to further explore the effects of lower concentrations of polysorbate 80 (Tween 80) and other surfactants (0.1% Poloxamer 188 (sold under the tradename of Pluronic F68 by BASF) and PEG 4600 (polyethylene glycol having an average molecular weight of 4600 was purchased from Aldrich) on the bioavailability of intratracheal (IT) administrated Proleukin and monomeric rhIL-2.

In this fifth study, as in the previous studies, male Sprague-Dawley rats were divided into treatment groups (4 rats/group). Each rat received a single 400 μg dose of rhIL-2. IT dosing was administered using the same procedure as described above for the other four studies. Blood samples of all rats were collected at predetermined time intervals and heparinized. Plasma from the blood samples was separated and analyzed by immunoassay for IL-2 concentration.

Monomeric formulations varied according to the type or amount of surfactant used with 230 mM arginine present in all monomeric formulations tested. Proleukin formulations differed as to whether polysorbate 80 (Tween 80) was absent or present at 0.1%. No arginine was used with the Proleukin formulations.

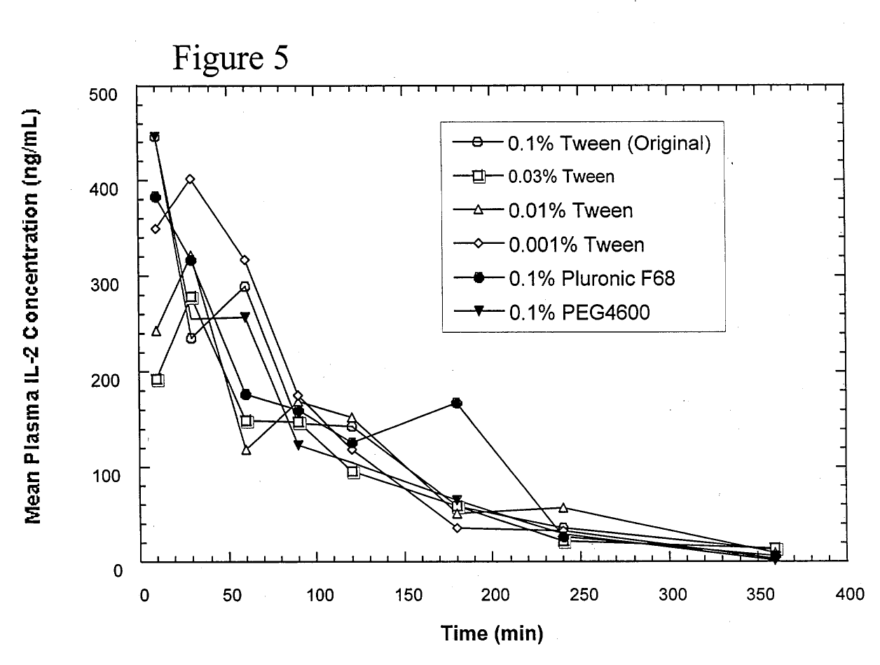

Plasma IL-2 concentration profiles from the studies are shown in FIG. 5. The area under the plasma concentration-time curve for IT dosing (AUC) for each formulation is shown in Table 5. The relative bioavailability of the Proleukin and monomeric formulations was calculated on a percentage basis using the AUC for the specific formulation compared to the AUC for monomeric with 0.1% polysorbate 80 (Tween 80) formulation as the reference and is shown in Table 5.

TABLE 5
Intratracheal bioavailability comparison among formulations at 400 μg
rhIL-2/animal.

| | AUC | Relative Bioavailability |
|---|---|---|
| Formulation | (ng-hr/ml) | (% to monomeric IT) |
| Monomeric (0.1% Tween 80)* | 664 | 100 |
| Monomeric (0.03% Tween 80) | 503 | 76 |
| Monomeric (0.01% Tween 80) | 637 | 96 |
| Monomeric (0.001% Tween 80) | 668 | 101 |
| Monomeric (0.1% Poloxamer 188) | 724 | 109 |
| Monomeric (0.1% PEG 4600) | 596 | 90 |
| Proleukin (no Tween 80) | 28.4 | 4 |
| Proleukin (0.1% Tween 80) | 86.2 | 13 |

*Reference formulation for calculating relative bioavailability.

The data above shows that the presence of polysorbate 80 may have played a role in the systemic absorption of IT-administered Proleukin. The data also indicate that bioavailability was similar for monomeric rhIL-2 formulated with Poloxamer 188, PEG 4600, 0.1% polysorbate 80 and 0.001% polysorbate 80. The bioavailability of the monomeric formulation containing 0.1% polysorbate 80 and arginine was higher compared to that of the Proleukin formulation with 0.1% polysorbate 80.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method for administering IL-2 to a subject in need thereof, said method comprising
a) obtaining a pharmaceutical composition comprising stabilized monomeric IL-2 or variants thereof;
b) preparing said composition as an aqueous or nonaqueous solution, an aqueous or nonaqueous suspension, or a dry powder form; and
c) delivering said solution, suspension, or dry powder form of said composition to said subject by pulmonary inhalation.

2. The method of claim 1, wherein said composition is in its liquid form.

3. The method of claim 1, wherein said composition is in its dried form, wherein said dried form is selected from the group consisting of a lyophilized form and a spray-dried form.

4. The method of claim 1, wherein said solution or suspension is delivered from a nebulizer or a metered-dose inhaler.

5. The method of claim 1, wherein said dry powder form is delivered from a metered-dose inhaler or a dry powder inhaler.

6. The method of claim 5, wherein said dry powder form consists of particles having a mean diameter less than 10 μm.

7. The method of claim 6, wherein said particles have a mean diameter in the range of 1 to 5 μm.

8. The method of claim 1, wherein said composition is a highly absorbable composition further comprising at least one surfactant in an amount sufficient to enhance absorption of said composition following pulmonary inhalation of said composition.

9. The method of claim 8, wherein said surfactant is selected from the group consisting of polysorbate 20 and polysorbate 80.

10. The method of claim 9, wherein said surfactant is polysorbate 80.

11. A method for administering IL-2 to a subject in need thereof, said method comprising:
a) obtaining a highly absorbable pharmaceutical composition comprising monomeric IL-2 or variants thereof, wherein said composition comprises at least one surfactant in an amount sufficient to enhance absorption of said composition following pulmonary inhalation of said composition;
b) preparing said composition as an aqueous or nonaqueous solution, an aqueous or nonaqueous suspension, or a dry powder form; and
c) delivering said solution, suspension, or dry powder form of said composition to said subject by pulmonary inhalation.

12. The method of claim 11, wherein said composition is in its liquid form.

13. The method of claim 11, wherein said composition is in its dried form, wherein said dried form is selected from the group consisting of a lyophilized form and a spray-dried form.

14. The method of claim 11, wherein said solution or suspension is delivered from a nebulizer or a metered-dose inhaler.

15. The method of claim 11, wherein said dry powder form is delivered from a metered-dose inhaler or a dry powder inhaler.

16. The method of claim 15, wherein said dry powder form consists of particles having a mean diameter less than 10 μm.

17. The method of claim 16, wherein said particles have a mean diameter in the range of 1 to 5 μm.

18. The method of claim 11, wherein said surfactant is selected from the group consisting of polysorbate 20 and polysorbate 80.

19. The method of claim 18, wherein said surfactant is polysorbate 80.

20. A method for administering IL-2 to a subject in need thereof, said method comprising
a) obtaining a pharmaceutical composition comprising multimeric IL-2 or variants thereof;
b) preparing said composition as an aqueous or nonaqueous solution, an aqueous or nonaqueous suspension, or a dry powder form; and
c) delivering said solution, suspension, or dry powder form of said composition to said subject by pulmonary inhalation.

21. The method of claim 20, wherein said composition is in its liquid form.

22. The method of claim 20, wherein said composition is in its dried form, wherein said dried form is selected from the group consisting of a lyophilized form and a spray-dried form.

23. The method of claim 20, wherein said solution or suspension is delivered from a metered-dose inhaler.

24. The method of claim 20, wherein said dry powder form is delivered from a metered-dose inhaler or a dry powder inhaler.

25. The method of claim 24, wherein said dry powder form consists of particles having a mean diameter less than 10 μm.

26. The method of claim 25, wherein said particles have a mean diameter in the range of 1 to 5 μm.

27. The method of claim 20, wherein said composition is a highly absorbable composition further comprising at least one surfactant in an amount sufficient to enhance absorption of said composition following pulmonary inhalation of said composition.

28. The method of claim 27, wherein said surfactant is selected from the group consisting of polysorbate 20 and polysorbate 80.

29. The method of claim 28, wherein said surfactant is polysorbate 80.

30. A method for administering IL-2 to a subject in need thereof, said method comprising
a) obtaining a pharmaceutical composition comprising a stabilized lyophilized or spray-dried IL-2 or variants thereof;
b) preparing said composition as an aqueous or nonaqueous solution, an aqueous or nonaqueous suspension, or a dry powder form; and
c) delivering said solution, suspension, or dry powder form of said composition to said subject by pulmonary inhalation.

31. The method of claim 30, wherein said solution or suspension is delivered from a nebulizer or a metered-dose inhaler.

32. The method of claim 30, wherein said dry powder form is delivered from a metered-dose inhaler or a dry powder inhaler.

33. The method of claim 32, wherein said dry powder form consists of particles having a mean diameter less than 10 μm.

34. The method of claim 33, wherein said particles have a mean diameter in the range of 1 to 5 μm.

35. The method of claim 30, wherein said composition is a highly absorbable composition further comprising at least one surfactant in an amount sufficient to enhance absorption of said composition following pulmonary inhalation of said composition.

36. The method of claim 35, wherein said surfactant is selected from the group consisting of polysorbate 20 and polysorbate 80.

37. The method of claim 36, wherein said surfactant is polysorbate 80.

38. A method for enhancing bioavailability of IL-2 administered to a subject via pulmonary inhalation, said method comprising administering a pharmaceutical composition comprising stabilized monomeric IL-2 or variants thereof to said subject via pulmonary inhalation.

39. The method of claim 38, wherein said composition further comprises at least one surfactant in an amount sufficient to enhance absorption of said composition following pulmonary inhalation of said composition.

40. A method for enhancing bioavailability of IL-2 administered to a subject via pulmonary inhalation, said method comprising administering a highly absorbable pharmaceutical composition comprising monomeric IL-2 or variants thereof to said subject via pulmonary inhalation, wherein said composition comprises at least one surfactant in an amount sufficient to enhance absorption of said composition following pulmonary inhalation of said composition.

41. A method for enhancing bioavailability of IL-2 administered to a subject via pulmonary inhalation, said method comprising administering a pharmaceutical composition comprising multimeric IL-2 or variants thereof to a subject via pulmonary inhalation, wherein said composition comprises at least one surfactant in an amount sufficient to enhance absorption of said composition following pulmonary inhalation of said composition.

42. A method for enhancing bioavailability of IL-2 administered to a subject via pulmonary inhalation, said method comprising administering a pharmaceutical composition comprising stabilized lyophilized or spray-dried IL-2 or variants thereof to a subject via pulmonoary inhalation, wherein said composition comprises at least one surfactant in an amount sufficient to enhance absorption of said composition following pulmonary inhalation of said composition.

43. A stabilized lyophilized or spray-dried pharmaceutical composition comprising:
interleukin-2 (IL-2) as a therapeutically active component;
at least one bulking agent present at about 0% to about 5%;
at least one of a sugar selected from the group consisting of sucrose, trehalose, raffinose, stachyose, sorbitol or at least one of an amino acid selected from the group consisting of arginine and lysine, wherein said sugar is present at about 0% to about 9%, and wherein said amino acid is present at about 0% to about 1%; and
a buffering agent to maintain said composition at a pH of about pH 4.0 to about pH 8.5 when said composition is reconstituted as a liquid.

44. A pharmaceutical composition comprising interleukin-2 (IL-2) or variants thereof as a therapeutic agent, wherein said composition comprises at least one surfactant in an amount sufficient to enhance absorption of said composition following pulmonary inhalation of said composition.

45. The composition of claim 44, wherein said surfactant is selected from the group consisting of polysorbate 20 and polysorbate 80.

46. A method for administering interleukin-2 (IL-2) or variants thereof to a subject in need thereof, said method comprising administering the composition of claim 44 via pulmonary inhalation.

47. The method of claim 45, wherein said composition is administered from a nebulizer or a metered-dose inhaler.

48. The method of claim 45, wherein said composition is administered from a metered-dose inhaler or a dry powder inhaler.

49. The method of claim 47, wherein said composition is in a dry powder form consisting of particles having a mean diameter less than 10 μm.

50. The method of claim 48, wherein said particles have a mean diameter in the range of 1 to 5 μm.
